(19) United States
(12) Patent Application Publication
(10) Pub. No.: US 2025/0311752 A1
(43) Pub. Date: Oct. 9, 2025

(54) METHOD OF ENHANCING IMMUNE-RESILIENCE OF FISH TO SKIN DISEASES AND SKIN PARASITES

(71) Applicant: Vaxa Technologies Ltd, Rosh Pina (IL)

(72) Inventor: Isaac BERZIN, Jerusalem (IL)

(73) Assignee: Vaxa Technologies Ltd, Rosh Pina (IL)

(21) Appl. No.: 19/240,976

(22) Filed: Jun. 17, 2025

Related U.S. Application Data
(62) Division of application No. 17/782,708, filed on Jun. 6, 2022.
(60) Provisional application No. 62/943,829, filed on Dec. 5, 2019.

(51) Int. Cl. A23K 20/158; A23K 10/22; A23K 10/30; A23K 50/80

(52) U.S. Cl. CPC A23K 20/158; A23K 10/22; A23K 10/30; A23K 50/80

(57) ABSTRACT

A nutritional supplement may be included in a fish diet and fed to fish in order to enhance immune-resilience of the fish to skin diseases and skin parasites. The nutritional supplement may include a carrier oil; 22-30 wt. % Omega 3; and 8-20 wt. % Omega 7. In some embodiments, the carrier oil may be a vegetable oil. In some embodiments, the Omega 3 includes EPA and DHA fatty acids. In some embodiments, the Omega 3 includes a mixture of EPA and DHA having ratios in the range of 1:10 to 10:1 wt. %. A method of enhancing immune-resilience of fish to skin diseases and skin parasites involves feeding to fish the nutritional supplement comprising a carrier oil, 22-30 wt. % omega 3 fatty acids and 8-20 wt. % omega 7 fatty acids.

### CROSS REFERENCE TO RELATED APPLICATIONS

This application is a divisional of U.S. patent application Ser. No. 17/782,708, filed Jun. 6, 2022, which is a US National Phase of PCT International Patent Application No. PCT/IL2020/051256, filed Dec. 6, 2020, which claimed the benefit of U.S. Provisional Patent Application No. 62/943,829, filed Dec. 5, 2019, the contents of each of which is hereby incorporated by reference in its entirety.

### FIELD OF THE INVENTION

The present invention relates to nutritional supplement for animal feed and aquaculture diet. More particularly, the present invention relates to nutritional supplement for animal feed and aquaculture diet that includes omega 3 and omega 7.

### BACKGROUND OF THE INVENTION

Nutritional supplements are known to increase the general wellbeing of animals and resilience to illnesses. Nutritional supplements can be added directly to the animal's ready-to-use dict or may be given as an additive in addition to the ready-to-use diet. For example, in fish, the addition of Omega-3's to the diet is known to increase the immune-resilience of the fish, in particular salmon. In addition to immune resilience to bacterial and viral infectious diseases and flatworm's, the immune system is important for resilience to parasite infestation, such as sea lice. Sea lice are parasitic copepods that cause large economic losses to salmon aquaculture worldwide. Frequent chemotherapeutic treatments are typically required to control this parasite, and alternative measures such as breeding for improved host resistance are desirable. Mechanical and chemical alterations induced by the sea lice parasite can have a profound effect on the host fish inflammatory and immune responses both directly (i.e. anti-coagulation, vasodilation, necrosis, etc.) and indirectly through stress induced immunosuppression.

Eicosapentaenoic acid (EPA) and docosahexaenoic acid (DHA) are omega-3 fatty acids (found in oily fish and microalgae). These fatty acids, and their metabolic derivatives-oxylipins, are bio-active molecules, able to inhibit aspects of inflammation including leucocyte chemotaxis, adhesion molecule expression and leucocyte-endothelial adhesive interactions, production of inflammatory cytokines and T cell reactivity. In parallel, EPA and DHA give rise to anti-inflammatory and inflammation resolving and protecting. Mechanisms underlying the anti-inflammatory actions of Omega-3 fatty acids include altered cell membrane phospholipid fatty acid composition, disruption of lipid rafts, inhibition of activation of the pro-inflammatory transcription factors-reducing expression of inflammatory genes, and activation of the anti-inflammatory transcription factors. The biological effects of oxylipins are mediated via interaction with receptors or intracellular effect.

While EPA and DHA are known to be effective nutritional supplement in salmons' diet, enabling limited relief form the ill effect of sea lice, EPA and DHA do not provide a complete shield against the sea lice infection. Accordingly, there is a need for a new nutritional supplement that may reduce the lice infection in a consistent, efficient and reliable way.

### SUMMARY OF THE INVENTION

Some aspects of the invention may be directed to a nutritional supplement that may be included in a fish diet. The nutritional supplement may include a carrier oil; 22-30 wt. % Omega 3; and 8-20 wt. % Omega 7. In some embodiments, the carrier oil may be a vegetable oil. In some embodiments, the Omega 3 includes EPA and DHA fatty acids. In some embodiments, the Omega 3 includes is a mixture of EPA and DHA having ratios in the range of 1:10 to 10:1 wt. %.

Some additional aspects of the invention may be directed to a ready to use diet, that may include: 0.7-7.5 wt. % Omega 3; 2.0-7.7 wt. % Omega 7; and a mixture comprising protein, carbohydrate and oil. In some embodiments, the protein may be 5-50 wt. % of the total weight of the ready to use fish diet. In some embodiments, the carbohydrate is 5-50 wt. % of the total weight of the ready to use diet. In some embodiments, the oil is 0.2-30 wt. % of the total weight of the ready to use fish diet. In some embodiments, the Omega 3 may include EPA and DHA fatty acids. In some embodiments, the carrier oil, may include at least one of: fish oil and vegetable oil. In some embodiments, the proteins may be originated from at least one of: Soy protein concentrate and animal byproducts

Some other aspects of the invention may be directed to a method of making a nutritional supplement. Embodiments of the method include, adding to a mixer, Omega 3 containing oil and Omega 7 containing oil; and mixing together the Omega 3 and Omega 7 containing oils. In some embodiments, the Omega 3 containing oil is at least one of: fish oil, Schizochytrium oil, GM Rapeseed oil and Anchovy oil. In some embodiments, the Omega 7 containing oil is sea Buckthorn oil. In some embodiments, the final amount of Omega 3 in the nutritional supplement is 0.22-30 wt. %. In some embodiments, the final amount of Omega 7 in the nutritional supplement is 8-20 wt. %.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows a flowchart of a method of making nutritional supplement according to some embodiments of the invention;

FIG. 2 is a graph showing feeding rates of juvenile salmons feed with standard a ready-to-use diet in comparison to feeding rates of juvenile salmons feed with a ready-to-use diet according to some embodiments of the invention . . .

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Some aspects of the invention may be directed to providing and/or making nutritional supplement that can increase the immune-resilience of animals, in particular fish to skin diseases and skin parasites. Such a nutritional supplement may include relatively high amounts of Omega 3 and Omega 7 fatty acids. A nutritional supplement according to some embodiments of the invention may be included in a ready-to-eat diet.

Omega 3 is a known nutritional supplement in fish diets, although having a limited immune-resilience effect. In some embodiments, it was found that an addition of Omega 7 to a nutritional supplement including Omega 3, enhance the effect of the Omega 3 and further to add to the general wellbeing of the fish skin, thus may increase the resilience of the fish to sea lice as discussed herein below.

A nutritional supplement according to some embodiments of the invention may include 22-30 wt. % Omega 3 and 8-20 wt. % Omega 7 in a carrier oil. The oil may be any edible oil, that is edible by a specific animal or group of animals. For example, the oil can be a vegetable oil, a fish oil and the like.

In some embodiments, the Omega 3 may include any combination of EPA and DHA fatty acids. In some embodiments, the Omega 3 may include a mixture of EPA to DHA ratios ranging from 1:10 to 10:1.

In some embodiments, the nutritional supplement may be included in a ready-to-use diet. Such ready-to-use diet may include 0.7-7.5 wt. % Omega 3, 2.0-4.0 wt. % Omega 7 and a mixture comprising protein, carbohydrate and oil. In some embodiments, the Omega 3 may include any combination of EPA and DHA fatty acids.

In some embodiments, the protein is 5-50 wt. % of the total weight of the ready to use fish diet. In some embodiments, the protein may be originated from at least one of: soy protein concentrate and animal byproducts.

In some embodiments, the carbohydrate is 5-50 wt. % of the total weight of the ready to use fish diet. In some embodiments, the oil is 0.2-30 wt. % of the total weight of the ready to use fish diet. In some embodiments, the oil may include at least one of: fish oil and vegetable oil.

An example of a fish diet according to some embodiments of the invention is given in table 1.

TABLE 1
a fish diet according to some embodiments of the invention

| Ingredient | Wt % inclusion in diet |
|---|---|
| Fish meal | 35-5 |
| Fish oil | 12-0 |
| Animal by-products | 33-10 |
| Corn gluten | 7-1.4 |
| Soy protein concentrate | 35-7 |
| Wheat gluten meal | 4.7-0.9 |
| Raw wheat | 23-10 |
| Vegetable oil | 27-6 |
| Omega 7 | 2-4 |
| Omega 3 (EPA + DHA) | 0.7-7.5 |

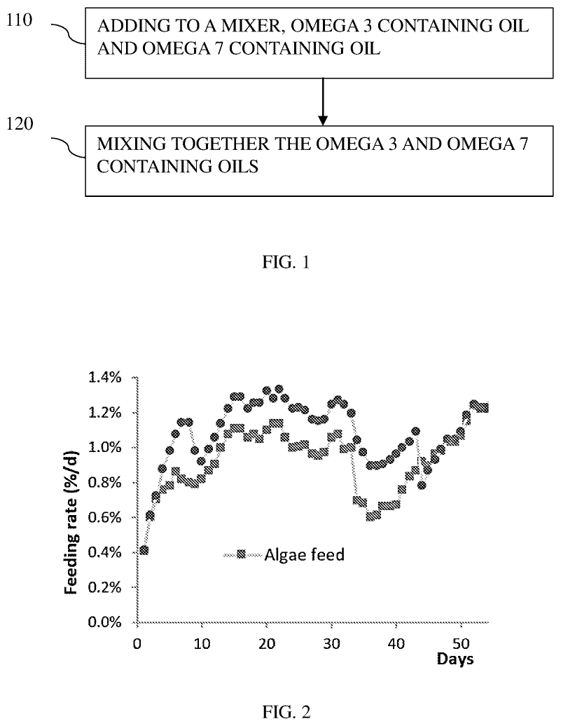

Reference is now made to FIG. 1 which is a flowchart of a method of making a nutritional supplement according to some embodiments of the invention. In step 110, Omega 3 containing oil and Omega 7 containing oil maybe added to a mixer. In some embodiments, the mixer may be any commercial mixer configured to mix oils, for example, the mixer may include mechanical stirrers, magnetic stirrers, ultrasonic stirrers and the like. Table 2 includes some examples for origins of Omega 3 containing oil and Omega 7 containing oil.

TABLE 2
origins of Omega 3 containing oil and Omega 7 containing oil

| Source | Wt. % Omega 7 | WL % EPA and DHA |
|---|---|---|
| Sea Buckthorn oil | 30 | 0 |
| Fish oil | 0 | 30 |
| Schizochytrium oil | 0 | 50 |
| GM Rapeseed oil | 0 | 30 |
| Anchovy oil | 6 | 25 |
| Nannochloropsis oil | 11 | 22 (EPA only) |

In some embodiments, the Omega 3 containing oil may include both DHA and EPA. In some embodiments, the method may include adding DHA to an EPA containing oil, for example, 50 wt. % DHA originated from Schizochytrium oil may be added to Nannochloropsis oil.

In step 120, the Omega 3 and Omega 7 containing oils may be mixed in the mixer. In some embodiments, the final amount of Omega 3 in the nutritional supplement may be 22-30 wt. %. In some embodiments, the final amount of Omega 7 in the nutritional supplement may be 8-20 wt. %.

### EXAMPLES

### Example 1

In some embodiments, mixing 100 g of Sea Buckthorn oil with 500 g of Anchovy oil yields 600 g of oil containing 57 g of Omega-7 and 150 g of Omega 3, which results in 11 wt. % Omega 7 and 25 wt. % Omega 3.

### Example 2

In some embodiments, mixing 150 g of Sea Buckthorn oil with 300 g of fish oil yields 450 of oil containing 45 g of Omega-7 and 90 g of Omega 3, which results in 10 wt. % Omega 7 and 20 wt. % Omega 3.

### Example 3

In some embodiments, mixing 100 g of GM rapeseed oil, containing 30 g Omega-3, with 100 g of Schizochytrium oil, containing 50 g Omega-3 and 100 g of Sea buckthorn oil, containing 30 g Omega-7, which results in 10 wt. % Omega-7 and 26.6 wt. % Omega-3.

### Example 4

In some embodiments, mixing 100 g Nannochloropsis oil, containing 11 g of Omega-7 and 22 g of EPA Omega-3, with 30 g of Schizochytrium oil, containing 9 g of Omega-3, which results in 8.5 wt. % Omega-7 and 24 wt. % Omega-3

### Experimental Results

### Experiment 1

The effect to adding both Omega 3 and Omega 7 to salmons' diet was studied. A nutritional supplement according to some embodiments of the invention was provided to salmons and compared to a first diet that includes Omega 3 and a second diet that did not include Omega 3 or Omega 7. Four 500-liter tanks having circular flow-through were used in this study, each was inhibited by 20 Atlantic Salmons. The temperature was 8.7° C. and the salinity 27.4 ppt. Each of the four tanks was provided with a different type of feed (e.g., diet).

The salmon in the first tank were fed with control, plant-based feed (denoted as [P]). The salmon in the second tank were fed with Omega 3 enriched diet (denoted as [P+O3]). The salmon in the third tank were fed with Omega 7 enriched diet (denoted as [P+O7]). The salmon in the fourth tanks were fed with a diet enriched with nutritional supplement according to some embodiments of the invention containing both Omega 3 and Omega 7 (denoted as [P+O3+O7]) form a combination of Schizochytrium and Nannochloropsis extract olis.

All the fish were first fed for 10 days with the control diet [P] and then each tank was provided with a different diet for additional 12 days. Fish from the four dietary groups were challenged with L. salmonis (50 copepodites per fish). Lice counting and sampling were done when majority of lice reached preadult stages (16-20 days after infection). During a sampling period of four days, number, stage and gender of lice on each fish were recorded. The average number of lice detected on the salmons in each tank is summarized in table 3.

TABLE 3
number of lice deleted on a salmon in each tank

| Feed | P | P + O3 | P + O7 | P + O3 + O7 |
|---|---|---|---|---|
| Sea lice per fish | 34 ± 3 | 20 ± 5 | 29 ± 4 | 7 ± 2 |
| (Ave ± STDV) | | | | |

As may be evident from table 3 the use of the nutritional supplement according to some embodiments of the invention provides the best protection against sea lice and significantly enhanced (˜80%) infestation reduction. As can be seen the addition of Omega 3 provides limited protection form sea lice infection, in comparison to the combined effect of Omega 3 and Omega 7.

Oxylipins function not only as biological “repellants” but are actively involved in a wide range of functions on the skin, mucous and cellular levels. Their biological effects is mediated via interaction with receptors or intracellular effectors. Omega 7, active in skin and mucous membranes, enhances the efficiency and functionality of these receptors, thus enhancing the immune response and effectivity of Oxylipins. It is also noticed that the copepodites reacted to the fish by swimming fast towards them, and once attached “made their decision” weather to stay on the host fish or not. In the case of the different experimental groups their decision varied.

### Experiment 2

The effect of a diet containing both Omega 3 and Omega 7, originated form algae oil, was tested on juvenile salmon smolts. When the salmon smolts reached mean weight of about 100 g they were put in six tanks having 1-meter dimeter. 200 Juvenile salmon smolts inhibited each tank. The juvenile salmon smolts in 3 tanks were fed with a ready-to-eat diet according to some embodiments of the invention and in the other 3 thanks were fed with commercial Juvenile salmon smolts. Tables 4 and 5 summarizes the rew materials and nutrition content and values of each diet.

TABLE 4
Raw material formulation of the diets as percentage (%)

| | Control | Algae |
|---|---|---|
| Raw materials: | diet | oil diet |
| Fishmeal 70 | 31.5 | 31.4 |
| Fish oil | 4.3 | 0.0 |
| Rapeseed oil | 17.2 | 14.1 |
| Algae oil | 0.0 | 8.4 |
| Wheat | 11.1 | 10.2 |
| SPC 61 | 16.1 | 16.1 |
| CGM61 | 4.0 | 4.0 |
| SBM Hipro 48 | 10.0 | 10.0 |
| VWG 82 | 0.2 | 0.2 |
| Rapeseed meal 29/11 | 4.0 | 4.0 |
| Premix—Laxá | 1.0 | 1.0 |
| Moncal-Phosphat | 0.3 | 0.3 |
| Panaferd colorant | 0.3 | 0.3 |

TABLE 5
Nutrient composition as gram per kg diet

| | Control | Algae |
|---|---|---|
| Nutrients: | diet | oil diet |
| Dry matter g/kg | | |
| Protein g/kg | 420.0 | 420.0 |
| Fat g/kg | 260.0 | 260.0 |
| Ash g/kg | 73.0 | 75.0 |
| Fiber g/kg | 19.0 | 19.0 |
| DHA g/kg | 7.3 | 2.7 |
| EPA g/kg | 6.2 | 24.3 |
| Sum EPA and DHA g/kg | 13.5 | 27.0 |

Reference is now made to FIG. 2 which shows the daily feeding rate of juvenile salmons fed with the control commercial diet (drack circles) in comparison to juvenile salmons fed with a diet according to embodiments of the invention. The initial daily feeding rate was lower for the salmons that were feed with algae enriched diet in comparison to the control diet. At later stages the feeding rate becomes similar for both feeds. This difference could be related to the fact that that the juvenile salmons had been fed normal salmon food in earlier stages prior to the experiment.

Prior to placing the salmons in the tanks 5 small fish and 5 larger fish were tested for fatty acid analysis of the flesh. Additional measurements were taken from each tank after 2 month of feeding and after 3 months of feeding. The results at the end of the process after 3 month of feeding are summarized in table 6.

TABLE 6
the percentage of fatty acids of total fatty acids measured in fish flash of fish fed
with diet according to embodiments of the invention and a control diet.

| | Algae-feed | | | Control feed | | | Av. | | |
|---|---|---|---|---|---|---|---|---|---|
| | Tank | Tank | Tank | Tank | Tank | Tank | Tanks | Av. Tanks | % |
| Composition | 1 | 2 | 3 | 4 | 5 | 6 | 1-3 | 4-6 | difference |
| SFA | 16.0 | 15.6 | 16.1 | 15.6 | 15.9 | 15.5 | 15.9 | 15.7 | 1% |
| MUFA | 51.7 | 51.5 | 51.4 | 53.2 | 52.8 | 52.7 | 51.5 | 52.9 | −3% |
| PUFA | 31.2 | 31.4 | 31.2 | 30.2 | 30.4 | 30.3 | 31.3 | 30.3 | 3% |
| unknown | 1.1 | 1.5 | 1.2 | 1.0 | 0.9 | 1.4 | 1.3 | 1.1 | 12% |
| EPA Omega 3 | 3.4 | 3.7 | 3.4 | 1.8 | 1.9 | 1.7 | 3.5 | 1.8 | 49% |
| DHA Omega 3 | 8.1 | 7.4 | 7.9 | 7.6 | 7.9 | 6.9 | 7.8 | 7.5 | 4% |
| Total Omega 3 | 17.8 | 17.7 | 17.7 | 15.0 | 15.5 | 14.6 | 17.7 | 15.0 | 15% |
| Palmitoleic acid | 4.7 | 4.8 | 4.8 | 2.4 | 2.4 | 2.4 | 4.8 | 2.4 | 49% |
| (Omega 7) | | | | | | | | | |

SFA = saturated fatty acids;
MUFA = monounsaturated fatty acids;
PUFA = polyunsaturated fatty acids;
TFA = trans fatty acids;
EPA = eicosapentaenoic acid;
DPA = docosapentaenoic acid;
DHA = docosahexaenoic acid;
PA = Palmitoleic acid Omega 7 (C16:1n-7)

As can clearly shown from the table a major increase in the amount of fatty acid was detected in the salmon fed with the diet according to embodiments of the invention. More specifically a major increase was presented in the amounts of Omega 3 and Omega 7.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention. Further, features or elements of different embodiments may be used with or combined with other embodiments.

1. A method of enhancing immune-resilience of fish to skin diseases and skin parasites comprising feeding to fish a nutritional supplement comprising a carrier oil, 22-30 wt. % omega 3 fatty acids and 8-20 wt. % omega 7 fatty acids.

2. The method of claim 1, wherein the carrier oil in the nutritional supplement is prepared from Nannochloropsis oil and/or Schizochytrium oil.

3. The method of claim 1, wherein the omega 3 fatty acids in the nutritional supplement include a mixture of eicosapentaenoic acid (EPA) and docosahexaenoic acid (DHA) fatty acids, having ratios in the range of 1:10 to 10:1 wt. % EPA:DHA.

4. The method of claim 1, wherein the carrier oil in the nutritional supplement is prepared by mixing a first carrier oil containing omega 3 fatty acids and a second carrier oil containing omega 7 fatty acids.

5. The method of claim 4, wherein the nutritional supplement further comprises fishmeal.

6. The method of claim 5, wherein the step of feeding the nutritional supplement to fish comprises feeding to fish a ready-to-use diet for fish containing the nutritional supplement, wherein the fishmeal includes a mixture comprising protein and carbohydrates.

7. The method of claim 6, wherein the ready-to-use diet for fish containing the nutritional supplement comprises 0.7-7.5 wt. % omega 3 fatty acids and 2.0-7.7 wt. % omega 7 fatty acids.

8. The method of claim 6, wherein the ready-to-use diet for fish containing the nutritional supplement comprises 5-50 wt. % protein, 5-50 wt. % carbohydrates and 0.2-30 wt. % oil of the total weight.

9. The method of claim 6, wherein the carrier oil comprises fish oil and/or vegetable oil, and wherein the protein comprises soy protein concentrate and/or animal byproducts.

10. The method of claim 6, wherein the carrier oil comprises oil from anchovy, rapeseed and/or sea buckthorn.

11. The method of claim 1, wherein the nutritional supplement is configured to enhance immune-resilience of fish against sea lice.

12. The method of claim 1, wherein the step of feeding the nutritional supplement to fish comprises feeding to fish a fish feed composition containing the nutritional supplement.
